(19) United States
(12) Patent Application Publication
(10) Pub. No.: US 2010/0114206 A1
(43) Pub. Date: May 6, 2010

(54) Patient Interface Device and Therapy Delivery System

(76) Inventors: William F. Kaemmerer, Edina, MN (US); Elizabeth A. Ellingson, Blaine, MN (US); Mark L. Brown, North Oaks, MN (US); Bruce D. Gunderson, Plymouth, MN (US); Holly Vitense, Maple Grove, MN (US)

Correspondence Address: Medtronic, Inc., 710 Medtronic Parkway, Mail Stop LC340, Minneapolis, MN, 55432, US

(21) Appl. No.: 12/263,279

(22) Filed: Oct. 31, 2008

(51) Int. Cl. A61N 1/39

(52) U.S. Cl. 607/5

(57) ABSTRACT

A method and system for diagnosing a medical condition, alerting a patient that a therapy is impending, and allowing the patient to override or modify the impending therapy are provided. The system may include an implantable medical device (“IMD”) and a patient interface device. The IMD may sense a physiological parameter within a patient, determine whether the physiological parameter qualifies for therapy based on a therapy criteria, and alert the patient via the patient interface device that therapy is impending if the physiological parameter qualifies for therapy. The patient may be given an opportunity to respond to the alert with the patient interface device and send an therapy modification indication if the therapy is not needed or wanted. The IMD may wait a predetermined period of time for the patient response and deliver the therapy if the patient response is not transmitted before expiration of the predetermined period of time or withhold the therapy if the patient overrides the delivery of the therapy within the predetermined period of time. The IMD may adjust one or more operational settings applied during its operation in response to the activity of the system. The patient interface device may be configured to avoid accidental override or modification of the delivery of the therapy.

### TECHNICAL FIELD

This disclosure relates generally to medical devices and more particularly to a system and method for diagnosing a medical condition, alerting a patient that a therapy is impending, and allowing the patient to override or modify the impending therapy.

### BACKGROUND

Historically, an implantable, subcutaneous or external medical device may be used to monitor physiological parameters in a patient to ensure that they fall within certain acceptable values or ranges. Such medical devices have further been capable of delivering therapy to a patient, where the device may be configured to automatically deliver the therapy in response to the monitored physiological parameters reaching certain values or ranges.

### SUMMARY

A method and system are provided for determining that therapy should be delivered to a patient by a medical device in response to sensed physiological parameters, alerting the patient that the therapy is impending, and allowing the patient to override or modify the impending therapy. In one or more embodiments, the method involves sensing a physiological parameter within a patient; determining whether the sensed physiological parameter qualifies for a therapy to be delivered to the patient based on a therapy criteria; alerting the patient that the therapy is impending if the sensed physiological parameter qualifies for therapy; determining whether a therapy modification indication is received that satisfies a modification criteria; and modifying delivery of the therapy to the patient for a predetermined period of time if the therapy modification indication is received and the modification criteria are satisfied. In one or more embodiments, the method may include notifying a remote recipient of the receipt of the therapy modification indication, wherein such notification includes information relating to the sensed physiological parameter. In one or more embodiments, the method may include adjusting the therapy criteria in response to the receipt of the therapy modification indication; and after the predetermined period of time passes, resuming the sensing of the physiological parameter within the patient and the therapy determination using the adjusted therapy criteria. The therapy criteria adjustment may include increasing a specificity of therapy criteria for determining whether the sensed parameter qualifies for therapy to be delivered to the patient based on the therapy criteria. The modification criteria may include whether the therapy to be delivered comprises a high voltage pulse to be applied to at least a portion of the patient's heart, whether a predetermined amount of therapy has already been delivered to the patient, or whether certain desired actions have been performed on a patient activator device for causing initiation of the therapy modification indication. In one or more embodiments, the method may be performed in response to an indication from the patient that the patient is experiencing a condition that qualifies for the therapy.

The system for diagnosing a medical condition, alerting a patient that a therapy is impending, and allowing the patient to override or modify the impending therapy may include an implantable medical device (“IMD”) and a patient interface device. In one or more embodiments, the IMD may comprise a sensor configured to sense a physiological parameter within a patient, a controller configured to determine whether the sensed physiological parameter qualifies for a therapy to be delivered to the patient based on a therapy criteria, a therapy module configured to deliver the therapy, and a telemetry module configured to send an alert when the therapy is impending. In one or more embodiments, the patient interface device may comprise a transceiver configured to receive the alert, an output device configured to notify the patient when the therapy is impending, an input device configured to receive a therapy modification indication from the patient, wherein the transceiver is further configured to send the therapy modification indication to the IMD. In one or more embodiments, the implantable medical device may be configured to modify delivery of the therapy to the patient for a predetermined period of time if the therapy modification indication is received and satisfies a modification criteria. In one or more embodiments, the implantable medical device may deliver a predetermined amount of therapy to the patient prior to allowing therapy to be modified by the therapy modification indication.

In one or more embodiments, the implantable medical device may include means for sensing a parameter within a patient; means for determining whether the sensed parameter qualifies for a therapy to be delivered to the patient based on a therapy criteria; means for alerting the patient that the therapy is impending if the sensed parameter qualifies for therapy; means for determining whether a therapy modification indication is received that satisfies a modification criteria; and means for modifying delivery of the therapy to the patient for a predetermined period of time if the therapy modification indication is received and the modification criteria are satisfied. In one or more embodiments, the implantable medical device may also include means for notifying a remote recipient of the receipt of the therapy modification indication, wherein such notification includes information relating to the sensed parameter; means for adjusting the therapy criteria in response to the receipt of the therapy modification indication; means for resuming the sensing of the parameter within the patient and the therapy determination using the adjusted therapy criteria after the predetermined period of time passes; or means for delivering a predetermined amount of therapy to the patient prior to allowing therapy to be modified by the therapy modification indication. In one or more embodiments, the modification criteria may include whether the therapy to be delivered comprises a high voltage pulse to be applied to at least a portion of the patient's heart or whether certain desired actions have been performed on a patient activator device for causing initiation of the therapy modification indication.

In one or more embodiments, the patient interface device may apply a unique identifier to the therapy modification indication to ensure that therapy modification indications are only received from a desired patient interface device. In one or more embodiments, the unique identifier may correspond to the associated implantable medical device. In one or more embodiments, the input device of the patient interface device may be configured to cause the therapy modification indication to be sent to the IMD only if a predetermined input is entered or a certain action is taken by the patient. In one or more embodiments, the input device may comprise a plurality of buttons and the predetermined input may require pressing each button in any sequence, in a predetermined sequence, or simultaneously. In one or more embodiments, the input device may comprise at least one button and the predetermined input may require continually pressing the at least one button until therapy is no longer impending.

### DRAWINGS

The above-mentioned features and objects of the present disclosure will become more apparent with reference to the following description taken in conjunction with the accompanying drawings wherein like reference numerals denote like elements and in which:

FIG. 1 illustrates components of the system including an implantable medical device in accordance with one or more embodiments of the present disclosure.

FIG. 2 is a schematic block diagram illustrating the various system components configured to operate in accordance with one or more embodiments of the present disclosure.

FIG. 3 is an operational flow diagram illustrating a process for allowing a patient to override or modify an automatically initiated therapy in accordance with one or more embodiments of the present disclosure.

FIG. 4 is an operational flow diagram illustrating a process for allowing a patient to override or modify an automatically initiated therapy in accordance with one or more embodiments of the present disclosure.

FIG. 5 is a block diagram illustrating the various system components including an implantable medical device and a patient interface device in accordance with one or more embodiments of the present disclosure.

### DETAILED DESCRIPTION

In the following detailed description of embodiments of the present disclosure, reference is made to the accompanying drawings in which like references indicate similar elements, and in which is shown by way of illustration specific embodiments in which the present disclosure may be practiced. These embodiments are described in sufficient detail to enable those skilled in the art to practice the present disclosure, and it is to be understood that other embodiments may be utilized and that logical, mechanical, electrical, functional, and other changes may be made without departing from the scope of the present disclosure. The following detailed description is, therefore, not to be taken in a limiting sense, and the scope of the present disclosure is defined only by the appended claims. As used in the present disclosure, the term “or” shall be understood to be defined as a logical disjunction and shall not indicate an exclusive disjunction unless expressly indicated as such or notated as “xor.”

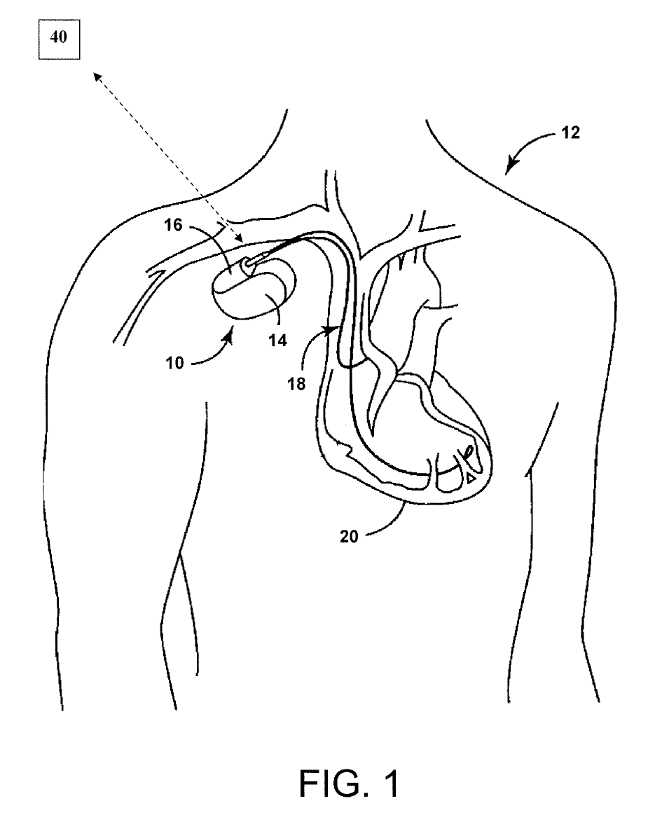

A method and system for diagnosing a medical condition, alerting a patient that a therapy is impending, and allowing the patient to override or modify the impending therapy are provided. In one or more embodiments, the system includes an implantable medical device (“IMD”) 10 and a patient interface device 40, as illustrated in the block schematic illustration of FIG. 1. FIG. 1 is a simplified schematic view of components of the system including an implantable medical device in accordance with one or more embodiments of the present disclosure. In one or more embodiments, IMD 10 may comprise a hermetically sealed enclosure 14 and connector module 16 for coupling IMD 10 to electrical leads and other physiological sensors arranged within body 12, such as pacing and sensing leads 18 connected to portions of heart 20 for delivery of pacing pulses to heart 20 of patient 12 and sensing at least one physiological parameter within heart 20. While IMD 10 is depicted in a pacemaker device configuration in FIG. 1, it is understood that IMD 10 may comprise any type of implanted, subcutaneous or external medial device. IMD 10 may collect and process data from one or more sensors for deriving parameters used in computing a probability that a medical condition is occurring in patient 12 in which IMD 10 is implanted. Patient interface device 40 may be capable of being arranged with respect to IMD 10 such that the various components are capable of communicating with each other.

In one or more embodiments, IMD 10 may be a device that is implanted within patient 12, as illustrated in FIG. 1. IMD 10 may be configured to sense a physiological parameter within patient 12. IMD 10 may be configured to deliver therapy to patient 12 based on the sensed physiological parameter. IMD 10 may be configured to alert patient 12 that delivery of therapy is impending prior to actual delivery of the therapy.

In one or more embodiments, IMD 10 may communicate with patient interface device 40. IMD 10 may send an alert to patient interface device 40 when therapy is impending. Patient 12 may operate patient interface device 40 to send a therapy modification indication to IMD 10. The therapy modification indication may override or modify the impending therapy and cause IMD 10 to modify the therapy for at least a predetermined period of time. In one or more embodiments, modification to a therapy may include overriding the therapy, delaying the therapy, modifying the way in which the therapy is delivered, modifying the amount of therapy delivered, exchanging the impending therapy for another therapy, or any other desired modification to the therapy that is impending. Patient interface device 40 may be readily accessible by patient 12. Patient interface device 40 may comprise an in-home monitoring device, a personal computer, a handheld or wearable device (e.g., a wristwatch), or any other device capable of receiving an input from patient 12 and communicating with other components of the system. Patient interface device 40 may be portable, carryable, wearable or located within the patient's home to allow patient 12 to receive alerts from IMD 10 or send therapy modification indications to IMD 10.

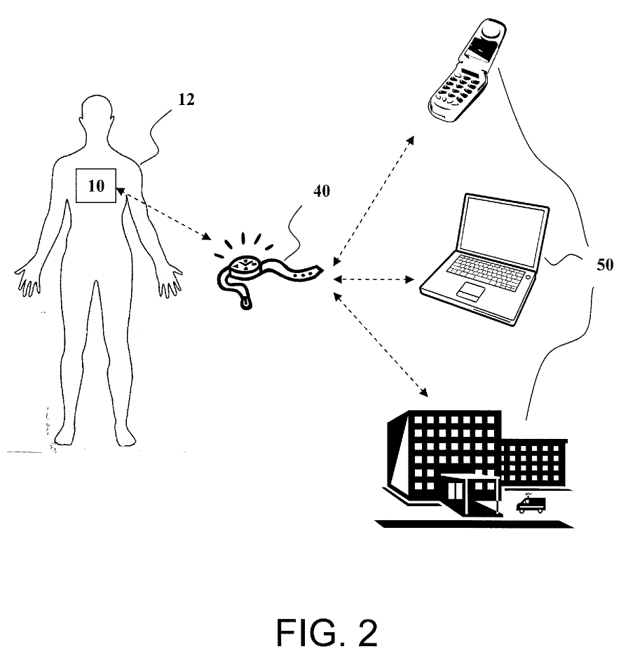

In one or more embodiments, patient interface device 40 may comprise a wearable watch that communicates wirelessly with IMD 10 implanted within patient 12, as illustrated in FIG. 2. In one or more embodiments, the system includes IMD 10, patient interface device 40, and remote recipient 50, as illustrated in the block schematic illustration of FIG. 2. In one or more embodiments, patient interface device 40 may communicate with at least one remote recipient 50. Patient interface device 40 may send data including at least a portion of: the operation history of patient interface device 40, the operation history of IMD 10, physiological parameters sensed by IMD 10, patient input provided to patient interface device 40, therapy delivered to patient 12, diagnostic procedures executed, or indications of a need for service to IMD 10 or patient interface device 40. Remote recipient 50 may be capable of analyzing the data received and returning a response comprising at least one of a confirmation signal, instructions for patient 12, and commands to be sent to IMD 10. In one or more embodiments, remote recipient 50 may be any device outside patient interface device 40 configured to receive the data from patient interface device 40. In one or more embodiments, remote recipient 50 may be a physician, a clinician, a portable device, a personal computer, or a facility where others may receive and analyze the data, as illustrated in FIG. 2. In one or more embodiments, the system may include an in-home monitoring device, such as the Medtronic CareLink® Network monitor, that collects information from IMDs implanted in patients and communicates such information to remote clinicians through the Internet, phone lines or wireless networks. CareLink is a registered trademark of Medtronic, Inc. of Minneapolis, Minn.

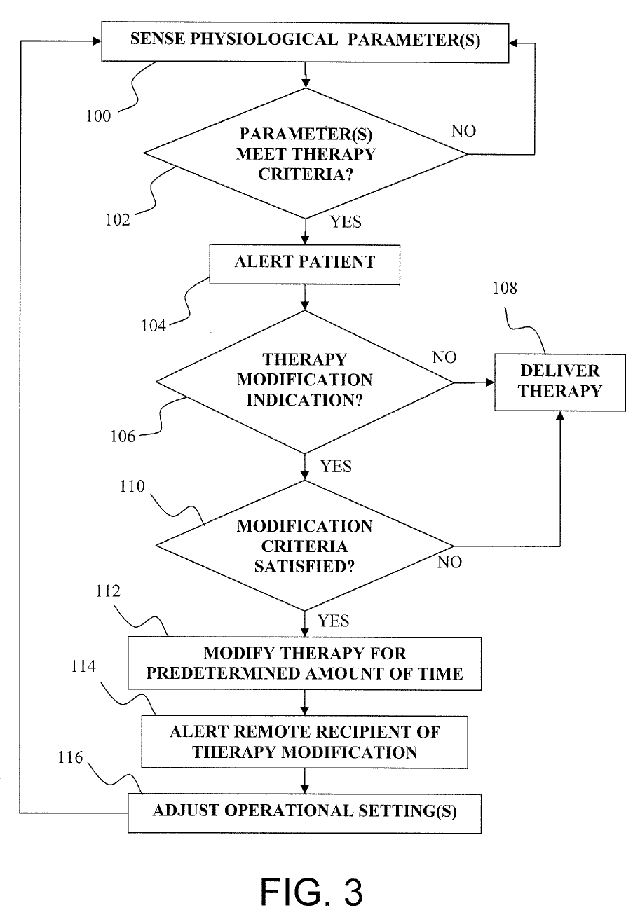

With reference to FIG. 3, an operational flow diagram is provided illustrating a process for allowing patient 12 to override or modify a therapy to be delivered by IMD 10 in accordance with one or more embodiments of the present disclosure. Initially, in operation 100, IMD 10 may sense one or more physiological parameters within patient 12. In one or more embodiments, physiological parameters may include cardiac activity, electrocardiograms, heart rate, pressure, and other metrics corresponding to events, activity, and conditions within patient 12.

In one or more embodiments, physiological parameters may include cardiac signals or events sensed by one or more sensors. In one or more embodiments, physiological parameters may include a set of separate measurements taken at different points in time or a waveform produced by a constant measurement across time. For example, such separate or constant measurements could be applied to algorithm-based analysis to detect whether the patient is experiencing a condition that qualifies for therapy.

In one or more embodiments, physiological parameters may include cardiac signals sensed by a plurality of sensors forming at least one sensing vector. The plurality of sensors may be located at different locations within patient 12. For example, a single sensing vector may be formed by a mathematical combination of measurements taken by each of the plurality of sensors. Such sensing vectors may aid in the estimation of physiological parameters.

In one or more embodiments, in operation 102, IMD 10 may determine whether a sensed physiological parameter satisfies one or more therapy criteria corresponding to whether the sensed physiological parameter qualifies for therapy. In one or more embodiments, therapy criteria may include a threshold determination of whether a sensed physiological parameter qualifies for therapy. In one or more embodiments, therapy criteria may include a lower limit, an upper limit, or a range within which a sensed physiological parameter may fall. In one or more embodiments, therapy criteria is an operational setting of IMD 10 that may be predetermined. In one or more embodiments, therapy criteria is an operational setting of IMD 10 that may be adjusted, as discussed further herein.

In one or more embodiments, therapy criteria may include detection of a condition that qualifies for therapy based on algorithm-based analysis of at least one sensed physiological parameter. For example, IMD 10 may apply a detection algorithm for combining or otherwise analyzing sensed cardiac events or electrogram (EGM) waveforms in order to detect whether a medical condition qualifying for therapy is present (e.g., ventricular fibrillation, myocardial infarction, heart failure, tachycardia, etc.). In one or more embodiments, IMD 10 may apply algorithms for selecting an appropriate therapy.

In one or more embodiments, if the sensed physiological parameter does not satisfy the therapy criteria, then IMD 10 may determine that the physiological parameter does not qualify for therapy and IMD 10 may resume physical parameter monitoring. In one or more embodiments, IMD 10 may immediately execute operation 100 after operation 102. In one or more embodiments, IMD 10 may wait for a predetermined period of time before executing operation 100 after operation 102.

In one or more embodiments, if the sensed physiological parameter does satisfy the therapy criteria, IMD 10 may alert patient 12, in operation 104, that the sensed physiological parameter qualifies for therapy and that therapy is impending. For example, in the situation where IMD 10 is an implantable cardioverter-defibrillator, the impending therapy may include a desired pulse to be delivered to the patient's heart. In one or more embodiments, IMD 10 transmits a signal to patient interface device 40, causing patient interface device 40 to notify patient 12 that therapy (e.g., a pulse) is impending. This provides patient 12 with an opportunity to modify therapy if patient 12 believes the therapy is unnecessary or undesired at that time.

In one or more embodiments, in operation 106, IMD 10 may determine whether patient 12 has provided a therapy modification indication. If patient 12 does not provide a therapy modification indication, then IMD 10 may proceed to operation 108, wherein IMD 10 may deliver therapy corresponding to the therapy criteria that has been satisfied. In one or more embodiments, patient 12 may communicate with IMD 10 by operating patient interface device 40. In one or more embodiments, patient interface device 40 may notify patient 12 of the results of the delivery of therapy.

In one or more embodiments, if patient 12 does provide a therapy modification indication, then in operation 110, IMD 10 may determine whether the therapy modification indication satisfies a modification criteria. Modification criteria of one or more embodiments are discussed below. If the therapy modification indication does not satisfy the modification criteria, then IMD 10 may proceed to operation 108, wherein IMD 10 may deliver therapy corresponding to the therapy criteria that has been satisfied. If the therapy modification indication does satisfy the modification criteria, then IMD 10 may proceed to operation 112, wherein it may modify therapy for a predetermined amount of time. In one or more embodiments, patient interface device 40 may notify patient 12 of the results of the modification of therapy.

In one or more embodiments, IMD 10 may wait for a predetermined period of time before delivering therapy, thereby providing the patient 12 with an opportunity to provide a therapy modification indication in response to the alert that therapy is impending. For example, if patient 12 has not provided a therapy modification indication before expiration of the predetermined period of time, then IMD 10 may deliver the therapy as planned in operation 108.

In one or more embodiments, the modification criteria verified in operation 110 may include whether a predetermined amount of therapy has been delivered to patient 12. In one or more embodiments the modification criteria may include whether a predetermined amount of therapy within a predetermined period of time has been delivered to patient 12. In one or more embodiments, the modification criteria may include whether a predetermined amount of therapy has been delivered for one or more of a predetermined number of sensed physiological parameters that qualify for therapy. For example, IMD 10 may deliver at least a minimal degree of therapy after a sensed physiological parameter is determined to qualify for therapy without allowing therapy to be modified, even if a therapy modification indication has been provided. After such minimal therapy is provided, patient 12 may be given an opportunity to override or modify any additional therapy to be subsequently provided. In one or more embodiments, the initial minimal therapy may be sensed or felt by patient 12 and may serve as the alert that is provided to patient 12 that additional therapy is impending.

In one or more embodiments, the modification criteria verified in operation 110 may include whether a predetermined amount of time has elapsed since the first sensed physiological parameter qualifying for therapy was sensed. In one or more embodiments, the modification criteria may include whether a predetermined number of sensed physiological parameters that qualify for therapy have been sensed. In one or more embodiments, the modification criteria may include whether a predetermined number of sensed physiological parameters that qualify for therapy have been sensed within a predetermined period of time. In one or more embodiments, the modification criteria may include whether a therapy modification indication was provided within a predetermined period of time.

In one or more embodiments, the modification criteria may include whether a sensed physiological parameter qualifies for a first type of therapy. In one or more embodiments, the first type of therapy may include a low energy therapy (e.g., pulse or shock that will not be painful to patient 12) such that there is no need to override or modify the low energy therapy. The sensed physiological parameter may, however, qualify for a different, second type of therapy, such as a high energy therapy (e.g., a high energy pulse, shock, or defibrillation that can be painful for patient 12). Patient 12 may need or desire to modify such second type of therapy if such painful therapy is not required. For example, a sensed physiological parameter that qualifies for high energy defibrillation therapy may not be necessary if patient 12 is not suffering from a medical condition that requires defibrillation (e.g., a life-threatening arrhythmia), and such a physiological parameter would satisfy the modification criteria. Furthermore, in this example, a sensed physiological parameter that qualifies for low energy pacemaking therapy or Cardiac Resynchronization Therapy (e.g., Bradycardia) may not satisfy the modification criteria, and such a low energy therapy may be delivered in operation 108 regardless of the patient's desire to modify such therapy.

In one or more embodiments, the modification criteria may include whether an activity within patient 12 qualifies to modify at least one therapy. In one or more embodiments, a modification criteria corresponding to an activity within patient 12 that is known to potentially interfere with delivery of a therapy, when satisfied, may cause a modification to any of the therapies. Such therapies could include any therapy deliverable by IMD 10, including high energy therapy, such as defibrillation, and low energy therapy, such as pacing for bradycardia, antitachycardia pacing (ATP) for tachycardias, or Cardiac Resynchronization Therapy. For example, where Cardiac Resynchronization Therapy offers a plurality of pacing vectors, and an activity (e.g., Phrenic Nerve Stimulation) is known to potentially interfere with the operation of one of the pacing vectors, the therapy may be modified such that only the pacing vectors that are not affected by any interference deliver therapy.

In one or more embodiments, the modification criteria may include whether a therapy modification indication received by IMD 10 from patient interface device 40 is valid, In one or more embodiments, the modification criteria may include whether the patient's condition has not significantly worsened in the time intervening between the detection of a potential need for therapy and the current time. In one or more embodiments, the modification criteria may include whether the therapy to be administered to the patient would be expected to be painful because it involves delivery of a high energy pulse to the heart.

In one or more embodiments, the modification criteria may include any combination of the modification criteria disclosed herein.

In one or more embodiments, in operation 114, patient interface device 40 may send data to remote recipient 50. The data sent may include at least a portion of: the operation history of patient interface device 40, the operation history of IMD 10, physiological parameters sensed by IMD 10, patient input provided to patient interface device 40, therapy delivered to patient 12, diagnostic procedures executed, or indications of a need for service to IMD 10 or patient interface device 40.

In one or more embodiments, in operation 116, IMD 10 may adjust one or more operational settings based upon the activity that occurred in previous operations. Operational settings that IMD 10 may adjust may include, but are not limited to: the manner in which physiological parameters are sensed, the manner in which events or conditions within patient 12 are detected (e.g. modify a detection algorithm and/or revert back to the initial therapy criteria), one or more therapy criteria, one or more modification criteria, a period of time for waiting to sense a physiological parameter, a period of time for waiting for a therapy modification indication, a type of therapy to be delivered, a remote recipient 50 to be alerted, a period of time for which modification of therapy applies, and/or any setting that affects the operation of IMD 10 and/or patient interface device 40.

In one or more embodiments, where sensed physiological parameters include a sensing vector based on cardiac signals sensed by a plurality of sensors, IMD 10 may adjust an operational setting corresponding to the formation of the sensing vector. For example, IMD 10 may alter which of the sensors contributes to the formation of the sensing vector or in what way the sensors contribute to the formation of the sensing vector (e.g., what weight each sensor will be given in a computation of an average value). In one or more embodiments, IMD 10 may adjust the formation of a sensing vector in response to a therapy modification indication from patient 12. For example, where IMD 10 senses a physiological parameter that satisfies a therapy criteria and patient 12 modifies therapy by responding to an alert, IMD 10 may adjust which sensors contribute to the sensing vector in order to achieve a more accurate measurement of a physiological parameter. For example, sensors involved in a therapy that is modified by patient 12 may be excluded from or given less weight in subsequent formulations of a sensing vector. In one or more embodiments, IMD 10 may perform diagnostic procedures to determine whether a given sensor is providing accurate measurements and thereby determine whether, or to what degree, the sensor should contribute to a sensing vector. In one or more embodiments, IMD 10 may communicate with a calibration device to determine which of various formations of sensing vectors provides the most accurate measurements. Such calibration devices may include patient interface device 40, remote recipient 50, or any other device that provides an accurate calibration standard and with which IMD 10 may communicate. For example, maintenance of IMD 10 by a medical professional may include calibration of a sensing vector. In one or more embodiments, IMD 10 may be manually configured to select a certain formation of a sensing vector according to known advantages. For example, where activity within patient 12 or imposed upon patient 12 is known by a medical professional to potentially interfere with the operation of certain sensors, a formation of a sensing vector that excludes or limits the contribution of such certain sensors may be selected.

In one or more embodiments, where IMD 10 senses a physiological parameter that satisfies a therapy criteria and patient 12 modifies therapy by responding to an alert, IMD 10 may adjust one or more operational settings, such as a therapy criteria, to reflect that the sensed physiological parameter does not qualify for therapy. For example, IMD 10 may increase the specificity applied when determining whether a sensed physiological parameter qualifies for therapy to be delivered to the patient based on a therapy criteria. In one or more embodiments, where IMD 10 senses a physiological parameter that satisfies a therapy criteria and patient 12 overrides therapy by responding to an alert, IMD 10 may adjust the period of time for waiting to sense a physiological parameter to reflect that sensing may be suspended for a longer period of time. In one or more embodiments, IMD 10 may perform diagnostic procedures to confirm the sensed physiological parameter. In one or more embodiments, IMD 10 may adjust one or more operational settings corresponding to a possible faulty operation of IMD 10. Where IMD 10 senses a physiological parameter that satisfies a therapy criteria and patient 12 overrides therapy by responding to an alert, IMD 10 may adjust one or more operational settings corresponding to diagnostic operations that are performed by IMD 10. For example, IMD 10 may perform diagnostic procedures to detect for possible malfunction of components of IMD 10, such as components relating to sensing a physiological parameter, or to detect interference from external influences, such as lead noise detection. Where faulty operation of IMD 10 is detected, IMD 10 may notify patient 12 or remote recipient 50 of a need for service via patient interface device 40. In one or more embodiments, an adjustment to one or more operational settings may be temporary, such that IMD 10 reverts to default physiological parameter sensing procedures after a predetermined period of time. In one or more embodiments, an adjustment to one or more operational settings may be fixed until a subsequent adjustment is made during a subsequent execution of operation 116.

In one or more embodiments, patient 12 may cause an adjustment to one or more operational settings prior to execution of operation 100 where IMD 10 may sense a physiological parameter within patient 12. In one or more embodiments, patient 12 may indicate to IMD 10 that patient 12 will be engaged in activity that justifies an adjustment to a therapy criteria. For example, patient 12 may indicate to IMD 10 that patient 12 will be exercising for a set duration of time, causing IMD 10 to adjust a therapy criteria to reflect that a sensed physiological parameter may be induced by exercise. In one or more embodiments, patient 12 may indicate, prior to operation 100, that sensing or delivery of therapy should be modified, such that patient 12 may modify or avoid therapy in situations where the patient's activities could provide misleading information regarding the sensed physiological parameters or during which therapy would be undesirable. In one or more embodiments, patient 12 may indicate to IMD 10 that patient 12 is experiencing an event or condition that qualifies for therapy by providing such indication through patient interface device 40. For example, where patient 12 is experiencing an event, such as pain or discomfort, that IMD 10 has not yet sensed, patient 12 may cause IMD 10 to adjust one or more operational settings, such as a period of time for waiting to sense a physiological parameter, such that IMD 10 may more promptly or accurately determine whether therapy should be delivered. In one or more embodiments, patient 12 may not adjust one or more operational settings until after a physiological parameter that qualifies for therapy has been sensed.

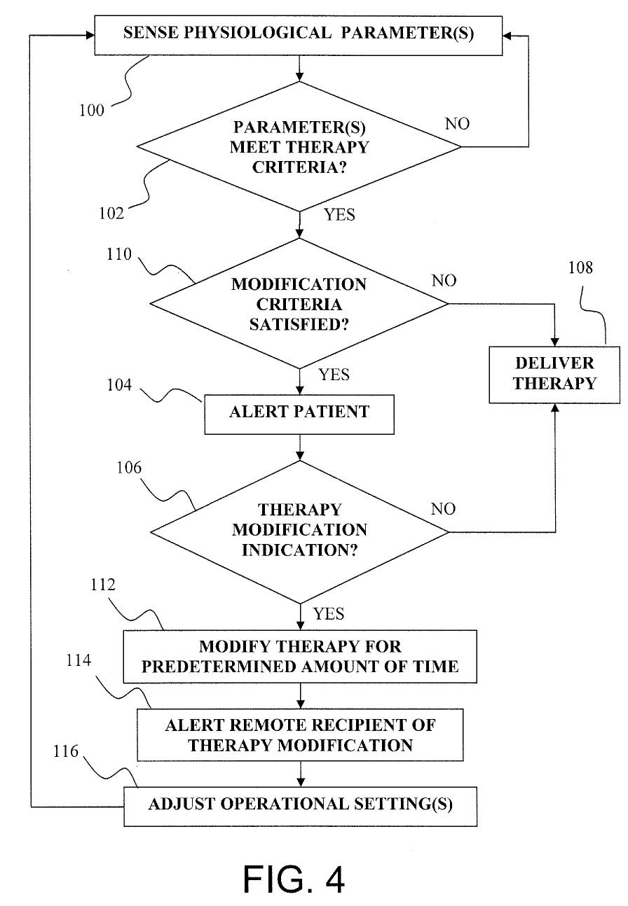

In one or more embodiments, the order in which the operations are executed as shown in FIG. 3 may vary. With reference to FIG. 4, the modification criteria verified in operation 110 may be applied after operation 102 where the determination of whether therapy should be applied is performed and before operation 104 where patient 12 is alerted of such impending therapy. For example, where a modification criteria is not satisfied, patient 12 may not be alerted and may not have an opportunity to override or modify the therapy that corresponds to the physiological parameter that satisfied the therapy criteria. For example, one aspect of the modification criteria applied in operation 110 may be that the impending therapy is known to be likely to cause pain or discomfort to the patient, and therefore, should not be delivered to the patient via operation 108 without first alerting the patient and providing the patient with an opportunity to provide a therapy modification indication.

In one or more embodiments, the modification criteria verified in operation 110 is alternatively applied after operation 104 where patient 12 is alerted of the impending therapy and before operation 106. In one or more embodiments, operations 112, 114, and 116 may be executed either substantially simultaneously or in any sequence. In one or more embodiments, one or more of operations 112, 114, and 116 may not be required to be executed and can be omitted. In one or more embodiments, any one of operations 112, 114, 116, or 100 may be performed before therapy is delivered in operation 108.

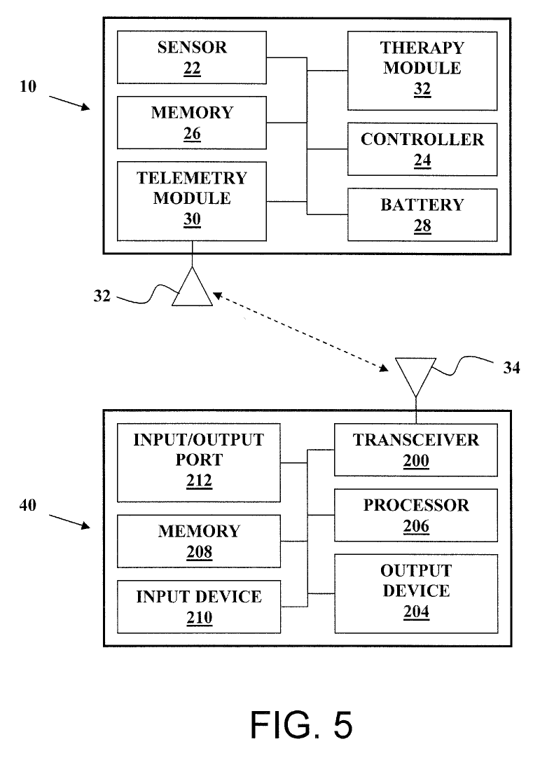

With reference to FIG. 5, a block diagram is provided illustrating the various system components including IMD 10 and patient interface device 40 in accordance with one or more embodiments of the present disclosure. IMD 10 is shown as including at least one sensor 22, controller 24, memory 26, battery 28, telemetry module 30, therapy module 32, and other components as appropriate to produce the desired functionalities of the device.

In one or more embodiments, sensor 22 may be configured to sense a physiological parameter within patient 12. IMD 10 may obtain data from physiological signals via electrodes and/or sensors 22 deployed on leads 18 and/or other sources. Sensors 22 may monitor electrical, mechanical, chemical, or optical information that contains physiological data of the patient and can utilize any source of physiological signals used for physiological events, parameters, or conditions. For example, sensor 22 may comprise a heart sensor, such as the MDT Reveal® system, commercially available from Medtronic of Minneapolis, that is capable of sensing cardiac activity, electrocardiograms, heart rate, pressure, or the like. Reveal is a registered trademark of Medtronic, Inc. of Minneapolis, Minn. In one or more embodiments, data obtained by sensor 22 may be provided to controller 24, which suitably analyzes the data, stores appropriate data in memory 26, or provides a response or report as appropriate.

In one or more embodiments, controller 24 may be configured to determine whether a sensed physiological parameter satisfies a therapy criteria. Controller 24 may be implemented with any type of microprocessor, digital signal processor, application specific integrated circuit (ASIC), field programmable gate array (FPGA) or other integrated or discrete logic circuitry programmed or otherwise configured to provide functionality as described herein. Controller 24 executes instructions stored in memory 26 to provide functionality as described herein. Instructions provided to controller 24 may be executed in any manner, using any data structures, architecture, programming language and/or other techniques. Memory 26 is any storage medium capable of maintaining digital data and instructions provided to controller 24 such as a static or dynamic random access memory (RAM), read-only memory (ROM), non-volatile random access memory (NVRAM), electrically erasable programmable read-only memory (EEPROM), flash memory, or any other electronic, magnetic, optical or other storage medium.

In one or more embodiments, communication between IMD 10 and patient interface device 40 can occur via telemetry, such as a long-distance telemetry system through the telemetry module 30. Telemetry module 30 may comprise any unit capable of facilitating wireless data transfer between IMD 10 and patient interface device 40, where patient interface device 40 may comprise an external medical device, a programming device, a remote telemetry station, a physician-activated device, a patient-activated device, a mobile handheld unit (e.g., mobile phone, PDA, etc.), a personal computer, an in-home monitoring device, a patient-wearable device, a display device or any other type of device capable of sending and receiving signals to and from IMD 10. Telemetry module 30 and patient interface device 40 may be respectively coupled to antennas 32 and 34 for facilitating wireless data transfer. Telemetry module 30 may be configured to perform any type of wireless communication. For example, telemetry module 30 may send and receive radio frequency (RF) signals, infrared (IR) frequency signals, or other electromagnetic signals. In the case of electromagnetic signals, antennas 32 and 34 may comprise coils for transmitting and receiving signals when positioned adjacent to one another. Any of a variety of modulation techniques may be used to modulate data on a respective electromagnetic carrier wave. Alternatively, telemetry module 30 may use sound waves for communicating data, or may use the patient's tissue as the transmission medium for communicating with a programmer positioned on the patients skin. Telemetry module 30 may facilitate wireless data transfer between IMD 10 and patient interface device 40. Other types of wired communications may also occur when IMD 10 is alternatively configured as an external medical device or contains wired communication channels that extend from within patient 12 to points outside of patient 12.

In one or more embodiments, therapy module 32 may be configured to deliver at least one type of therapy to patient 12. For example, therapy module 32 may produce a stimulus to the heart tissue of patient 12 (e.g., defibrillation stimulus, pacing stimulus, pacing rate adjustment, pacing characteristic adjustment, and/or pulse adjustment stimulus). In one or more embodiments, therapy module 32 may include a pacemaker, which may utilize electrical impulses to regulate the beating of the heart to treat bradycardia (a slow, irregular, or interrupted heartbeat). In one or more embodiments, therapy module 32 may include a biventricular pacemaker, also known as a cardiac resynchronization device, which may help selected chambers of the heart to beat in a more coordinated fashion, so that blood pumps more efficiently to the body. In one or more embodiments, therapy module 32 may include an implantable cardioverter defibrillator, which may provide burst pacing pulses or a defibrillation shock to the heart when the heart is beating too fast or goes into fibrillation.

With further reference to FIG. 5, patient interface device 40 is shown as including transceiver 200, output device 204, processor 206, memory 208, input device 210, input/output port 212, and other components as appropriate to produce the desired functionalities of the device.

In one or more embodiments, patient interface device 40 may include antenna 34, coil or wired input for communicating data and other signals between patient interface device 40 and IMD 10. Data may be received from IMD 10 through antenna 34, which is connected to transceiver 200 that may serve to receive and transmit communication signals through antenna 34. The demodulated signals may be applied in parallel or serial digital format to input/output (I/O) port 212, where they in turn may be applied to an output device 204 or provided to processor 206 or memory 208. In one or more embodiments, output device 204 may include a screen or types of interface devices capable of communicating information to the patient (e.g., an audio speaker device, a tactile vibrating device, or other output device). In one or more embodiments, output device 204 may be configured to alert patient 12 that therapy from IMD 10 is impending.

In one or more embodiments, patient interface device 40 may include an input device 210 that allows data, commands or selections to be input into patient interface device 40 by a patient, physician or clinician. For example, the patient may use input device 210 to enter a command to override or modify the therapy from IMD 10. Input device 210 may include, but is not limited to, at least one of the following: a keyboard, keypad, track ball, mouse, touch-sensitive displays, push buttons, magnetic readers, RF readers, tablets, styluses, microphones, voice recognizers, handwriting recognizers and any other device that allows a patient, physician or clinician to input data to external device. In one or more embodiments, patient interface device 40 will only send a therapy modification indication to IMD 10 when a predetermined input is provided by patient 12 into input device 210. For example, in one or more embodiments, input device 210 may comprise a plurality of buttons, wherein patient 12 must press each one of the plurality of buttons in any sequence to cause an therapy modification indication to be sent to IMD 10. In one or more embodiments, input device 210 may comprise a plurality of buttons, wherein patient 12 must press a predetermined sequence of buttons to cause an therapy modification indication to be sent to IMD 10. In one or more embodiments, input device 210 may comprise a plurality of buttons, wherein patient 12 must press multiple buttons simultaneously to cause an therapy modification indication to be sent to IMD 10. In one or more embodiments, input device 210 may comprise at least one button, wherein patient 12 must continually press the at least one button as long as therapy from IMD 10 is impending to cause an therapy modification indication to be sent to IMD 10.

In one or more embodiments, a signal containing data may be sent from patient interface device 40 to IMD 10. In one or more embodiments, patient interface device 40 may generate a signal with transceiver 200 to be received wirelessly by telemetry module 30 of IMD 10. In one of more embodiments, a given patient interface device 40 may be configured to be associated with a given IMD 10, such that the given patient interface device 40 may only communicate with the given IMD 10. For example, patient interface device 40 and IMD 10 may apply unique identifiers to signals sent that cause only an associated device to recognize and respond to the signals. In one or more embodiments, the unique identifier may correspond to a serial number of each associated device. For example, patient interface device 40 may apply the serial number of an associated IMD 10 to a signal to ensure that only the associated IMD 10 may respond to the signal. In one or more embodiments, a signal may employ an encryption/decryption scheme, wherein the signal is encrypted such that only an associated device is capable of decryption to interpret and respond to the signal. In one or more embodiments, a signal may contain data comprising the unique identifier and a command, such that the associated device may determine whether it is the intended recipient of the signal by reading the unique identifier and respond accordingly.

Processor 206 of patient interface device 40 may include any type of microprocessor, digital signal processor, application specific integrated circuit (ASIC), field programmable gate array (FPGA) or other integrated or discrete logic circuitry programmed or otherwise configured to control operating of patient interface device 40 and provide functionality as described herein. In one or more embodiments, processor 206 executes instructions stored in memory 208 to provide functionality as described herein. In one or more embodiments, instructions may be stored in memory 208 for operating a patient override program that allows patient 12 to override or modify the delivery of therapy by IMD 10. In one or more embodiments, data may be recorded in memory 208 for later transmission during communication with remote recipient 50. Such data may include at least a portion of: the operation history of patient interface device 40, the operation history of IMD 10, physiological parameters sensed by IMD 10, patient input provided to patient interface device 40, therapy delivered to patient 12, diagnostic procedures executed, or indications of a need for service to IMD 10 or patient interface device 40. Processor 206 may control operation of output device 204 and may be responsive to commands received from input device 210. Memory 208 is suitable for storing data received from IMD 10 or other sources, input device 210, processor 206 or other data or commands otherwise received by patient activator device 40. Patient interface device 40 may further include an input/output port 212 for connecting patient interface device 40 to other devices, communication networks, phone lines, wireless devices, etc. When data received from IMD 10 through the telemetry uplink is to be transmitted to a remote recipient 50 for further analysis, such information and data may be transmitted through input/output port 212 to a connected network or through transceiver 200 or to a wirelessly connected device.

While the system and method have been described in terms of what are presently considered to be specific embodiments, the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
sensing a parameter within a patient;
determining whether the sensed parameter qualifies for a therapy to be delivered to the patient based on a therapy criteria;
alerting the patient that the therapy is impending if the sensed parameter qualifies for therapy;
determining whether a therapy modification indication is received that satisfies a modification criteria; and
modifying delivery of the therapy to the patient for a predetermined period of time if the therapy modification indication is received and the modification criteria are satisfied.

2. The method of claim 1, further comprising notifying a remote recipient of the receipt of the therapy modification indication, wherein such notification includes information relating to the sensed parameter.

3. The method of claim 1, further comprising:
adjusting the therapy criteria in response to the receipt of the therapy modification indication; and
after the predetermined period of time passes, resuming the sensing of the parameter within the patient and the therapy determination using the adjusted therapy criteria.

4. The method of claim 1, wherein the modification criteria includes whether the therapy to be delivered comprises a high voltage pulse to be applied to at least a portion of the patient's heart.

5. The method of claim 1, wherein the modification criteria includes whether certain desired actions have been performed on a patient activator device for causing initiation of the therapy modification indication.

6. The method of claim 3, wherein the therapy criteria adjustment comprises increasing a specificity of therapy criteria for determining whether the sensed parameter qualifies for therapy to be delivered to the patient based on the therapy criteria.

7. The method of claim 1, further comprising delivering a predetermined amount of therapy to the patient prior to allowing therapy to be modified by the therapy modification indication.

8. The method of claim 1, wherein the method is performed in response to an indication from the patient that the patient is experiencing an event that qualifies for the therapy.

9. A system comprising:
an implantable medical device comprising a sensor configured to sense a parameter within a patient, a controller configured to determine whether the sensed parameter qualifies for a therapy to be delivered to the patient based on a therapy criteria, a therapy module configured to deliver the therapy, and a telemetry module configured to send an alert when the therapy is impending;
a patient interface device comprising a transceiver configured to receive the alert, an output device configured to notify the patient when the therapy is impending, an input device configured to receive a therapy modification indication from the patient, wherein the transceiver is further configured to send the therapy modification indication to the implantable medical device; and
wherein the controller of the implantable medical device is further configured to modify delivery of the therapy to the patient for a predetermined period of time if the therapy modification indication is received and a modification criteria are satisfied.

10. The system of claim 9, wherein the modification criteria includes whether the therapy to be delivered comprises a high voltage pulse to be applied to at least a portion of the patient's heart.

11. The system of claim 9, wherein the modification criteria includes whether certain desired actions have been performed on a patient activator device for causing initiation of the therapy modification indication.

12. The system of claim 9, wherein the controller of the implantable medical device is further configured to adjust the therapy criteria in response to the receipt of the therapy modification indication, and, after the predetermined period of time passes, resume the sensing of the parameter within the patient and the therapy determination using the adjusted therapy criteria.

13. The system of claim 12, wherein the controller of the implantable medical device is further configured to adjust the therapy criteria by increasing a specificity of therapy criteria for determining whether the sensed parameter qualifies for therapy to be delivered to the patient based on a therapy criteria.

14. The system of claim 9, wherein the implantable medical device is configured to deliver a predetermined amount of therapy to the patient prior to allowing therapy to be modified by the therapy modification indication.

15. An implantable medical device, comprising:
means for sensing a parameter within a patient;
means for determining whether the sensed parameter qualifies for a therapy to be delivered to the patient based on a therapy criteria;
means for alerting the patient that the therapy is impending if the sensed parameter qualifies for therapy;
means for determining whether a therapy modification indication is received that satisfies a modification criteria; and
means for modifying delivery of the therapy to the patient for a predetermined period of time if the therapy modification indication is received and the modification criteria are satisfied.

16. The implantable medical device of claim 15, further comprising means for notifying a remote recipient of the receipt of the therapy modification indication, wherein such notification includes information relating to the sensed parameter.

17. The implantable medical device of claim 15, further comprising:
means for adjusting the therapy criteria in response to the receipt of the therapy modification indication; and
means for resuming the sensing of the parameter within the patient and the therapy determination using the adjusted therapy criteria after the predetermined period of time passes.

18. The implantable medical device of claim 15, wherein the modification criteria includes whether the therapy to be delivered comprises a high voltage pulse to be applied to at least a portion of the patient's heart.

19. The implantable medical device of claim 15, wherein the modification criteria includes whether certain desired actions have been performed on a patient activator device for causing initiation of the therapy modification indication.

20. The implantable medical device of claim 15, further comprising means for delivering a predetermined amount of therapy to the patient prior to allowing therapy to be modified by the therapy modification indication.
